(19) United States
(12) Patent Application Publication
(10) Pub. No.: US 2004/0077832 A1
(43) Pub. Date: Apr. 22, 2004

(54) Jfy1protein induces rapid apoptosis

(76) Inventors: Jian Yu, Pittsburgh, PA (US); Kenneth W. Kinzler, Bel Air, MD (US); Bert Vogelstein, Baltimore, MD (US)

Correspondence Address: BANNER & WITCOFF, 1001 G STREET N W, SUITE 1100, WASHINGTON, DC, 20001, US

(21) Appl. No.: 10/450,436

(22) PCT Filed: Dec. 12, 2001
(86) PCT No.: PCT/US01/47455

(51) Int. Cl. A61K 48/00; A61K 38/17; C07K 14/47; C12Q 1/68; G01N 33/574; C07H 21/04

(52) U.S. Cl. 530/350; 435/7.23; 435/69.1; 435/320.1; 435/325; 514/12; 514/44; 536/23.5; 435/6

(57) ABSTRACT

Through global profiling of genes that were expressed soon after p53 expression, we identified a gene termed (JFY1). The protein encoded by (JFY1) was found to be exclusively mitochondrial and to bind to Bcl-2 and Bcl-X_{L} through a BH3 domain. Exogenous expression of (JFY1) resulted in an extremely rapid and profound apoptosis that occurred much earlier than that resulting from exogenous expression of p53. Based on its unique expression patterns, p53-dependence, and biochemical properties,(JFY1) is likely to be a direct mediator of p53-associated apoptosis.

This application claims the benefit of U.S. application Ser. No. 60/256,328, filed 19 Dec. 2000.

This invention was made using funds from the U.S. Government. The U.S. Government retains certain rights in the invention according to the provisions of NIH grants CA 43460 and GM 07184.

### BACKGROUND OF THE INVENTION

Inactivation of the growth-controlling functions of p53 appears to be critical to the genesis of most human cancers (Hollstein et al., 1999; Hussain and Harris, 1999). The p53 protein controls tumor growth by inhibiting cell cycle progression and by stimulating apoptosis (Lane, 1999; Levine, 1997; Oren, 1999; Prives and Hall, 1999). It has been shown that the inhibition of cell cycle progression by p53 is in large part due to its ability to transcriptionally activate genes that directly control cyclin-dependent kinase activity (reviewed in (El-Deiry, 1998)). For example, p53 induces p21^{CIP1/WAF1}, which binds to and inhibits several cyclin-cdk complexes (Harper et al., 1993; Xiong et al., 1993), and 14-3-3σ, which sequesters cyclin B/cdc2 complexes in the cytoplasm (Chan et al., 1999). In both cases, the induction results from p53 binding to cognate recognition elements in the promoters of these genes (El-Deiry et al., 1993; Hermeking, 1997).

Much less is known about the mechanisms through which p53 induces apoptosis, though this is also thought to be mediated by transcriptional activation of target genes (reviewed in (Chao et al., 2000)). The apoptotic function of p53 is highly conserved, as is evident from functional studies of the Drosophila p53 homolog (Brodsky et al., 2000; Jin et al., 2000; Ollmann et al., 2000). Moreover, the cell cycle inhibitory effects of p53 are inadequate to fully account for the tumor suppressor effects of p53, suggesting that apoptotic induction is a key component of p53's tumor suppression (Gottlieb and Oren, 1998; Symonds et al., 1994). Many studies have been performed to identify genes that are regulated by p53 and mediate apoptosis (El-Deiry, 1998). Among these candidates, those that encode mitochondrial proteins are particularly attractive because p53-initiated apoptosis appears to proceed through a mitochondrial pathway. In particular, the apoptosis stimulated by p53 involves disruption of mitochondrial membrane potential, accumulation of reactive oxygen species, stimulation of caspase 9 activity and subsequent activation of a caspase cascade (Li et al., 1999; Polyak et al., 1997; Schuler et al., 2000; Soengas et al., 1999).

Three genes that are regulated by p53 and encode proteins that at least partly reside in the mitochondria have been identified. The first to be identified was BAX, the pro-apoptotic Bcl-2 family member that serves as the prototype for this class (Reed, 1999). More recently, Noxa and p53AIP1 have been discovered and shown to encode pro-apoptotic mitochondrial proteins whose expression is controlled by p53 (Oda et al., 2000a, Oda, 2000b). To explore the role of these genes in colorectal cancers (CRC), we examined their expression patterns in detail. As described below, these three genes did not appear to be expressed at early enough times or at sufficiently robust levels to account for the dramatic apoptosis induced by p53 in CRC cells. There is a continuing need in the art for identification of genes which are involved in the induction of apoptosis of cancer cells.

### SUMMARY OF THE INVENTION

It is an object of the invention to provide an isolated and purified protein suitable for inducing rapid apoptosis in cancer cells.

It is an object of the invention to provide an isolated and purified polynucleotide encoding a protein suitable for inducing rapid apoptosis in cancer cells.

It is still another object of the invention to provide an isolated and purified nucleic acid containing a binding site for p53.

It is yet another object of the invention to provide a method of inducing apoptosis in cancer cells.

It is still another object of the invention to provide a method of screening drugs for those which can induce apoptosis.

It is an object of the invention to provide a method for diagnosing cancer cells.

It is another object of the invention to provide a method to aid in determining prognosis of a cancer patient.

These and other objects of the invention are provided by one or more of the embodiments described below. In one embodiment of the invention an isolated and purified JFY1 protein having the sequence shown in SEQ ID NO: 1 or 2 is provided.

In another embodiment of the invention an isolated and purified JFY1 polynucleotide is provided. It comprises a coding sequence having the sequence shown in SEQ NO: 3 or 4.

In yet another embodiment of the invention an isolated and purified JFY1 BS1 or BS2 nucleic acid is provided. It has the sequence shown in SEQ ID NO: 5, 6, or 27.

According to another aspect of the invention a method of inducing apoptosis in cancer cells is provided. A nucleic acid comprising a JFY1 coding sequence is supplied to cancer cells. JFY1 is thereby expressed and induces apoptosis in said cancer cells.

According to another aspect of the invention a method of screening drugs for those which can induce apoptosis is provided. A test compound is contacted with a cell comprising a mutant p53 and no wild-type p53. JFY1 expression is detected in the cell. A test compound which increases JFY1 expression is a candidate drug for treating cancer.

According to still another aspect of the invention a method of screening drugs for those which can induce apoptosis is provided. A test compound is contacted with a cell comprising a mutant p53 and a JFY1-BS2-reporter gene construct. The cell comprises no wild-type p53. Reporter gene expression is detected. A test compound which increases reporter gene expression is a candidate drug for treating cancer.

In another embodiment of the invention a method for diagnosing cancer cells is provided. An expression product of JFY1 is assayed in a biological sample suspected of being neoplastic. The amount of the expression product in the biological sample is compared to the amount of the expression product in a control sample which is not neoplastic. The biological sample is identified as neoplastic if the amount of the expression product in the biological sample is significantly less than the amount in the control sample.

In still another embodiment of the invention a method to aid in determining prognosis of a cancer patient is provided. An expression product of JFY1 is assayed in a tumor sample. The amount of the expression product in the tumor sample is compared to amount of the expression product in a control sample which is not neoplastic. The biological sample is identified as having a negative prognostic indicator if the amount of the expression product in the tumor sample is significantly less than the amount in the control sample.

Thus the present invention provides the art with a new gene and protein which are important in mediating p53 induced apoptosis in cancer cells.

### BRIEF DESCRIPTION OF THE DRAWINGS

FIGS. 1A to 1C. Induction of JFY1 by p53 in CRC cells. (FIG. 1A) Northern blot analyses of RNA samples prepared from p53-inducible DLD1 cells at the indicated time points are shown. The JFY1 gene was induced as early as 3 hours after doxycycline removal, similar to that of p21, while the BAX and Noxa genes were not induced as robustly. pS3AIP1 transcripts were not detectable under these conditions. A GAPDH probe was used as a loading control. (FIG. 1B) RNA from the indicated colorectal cancer cells lines infected with adenovirus expressing wt p53 (W) and mutant p53R75H (M) for 17 hours were analyzed by Northern blotting. (FIG. 1C) RNA from the indicated colorectal cancer cells lines treated with adriamycin (Adr) or 5-Fluorouracil (5-FU) for 24 hours was analyzed by Northern blotting. RNA from untreated cells (“Un”) was used as a control.

FIGS. 2A to 2B. The JFY1 protein contains a BH3 domain. (FIG. 2A) Alignment of the predicted amino acids of human (SEQ ID NO: 1) and mouse (SEQ ID NO:2) JFY1 reveals 90% identity. The identical residues are colored blue and non-conserved residues are colored red. The residues comprising AA128-165 were predicated to form an α-helix by the Chou-Fasman method. The middle third of the α-helix corresponding to the BH3 (AA141-149) domain is completely identical in both human and mouse JFY1. (FIG. 2B) Alignment of BH3 domains of JFY1 with other Bcl-2 family members. (SEQ ID NO:7-17) Conserved residues (contained in more than three members of the eleven shown) are colored blue, whereas the non-conserved residues are colored red.

FIGS. 3A to 3D. p53 activates the JFY1 promoter (FIG. 3A) The two potential p53 binding sites (BS1 and BS2; SEQ ID NOs: 5 and 6) within 300 bp of the putative transcription start site are indicated. The predicted open reading frame (ORF) starts at the indicated ATG. Frag1 and Frag2 were used in reporter constructs. The previously characterized p53-consensus binding site (CBS; SEQ ID NO:18) (El-Deiry et al., 1992) is shown above the BS1 sequence, with R=purine, Y=pyrimidine, and W=A or T. (FIG. 3B) The indicated fragments were cloned into pBVLuc and cotransfected into H1299 cells together with a wt (wt) or mutant (R175H) p53 expression construct (Baker et al., 1990). The ratio of luciferase activity in the presence of wt p53 compared to that in the presence of mutant p53 is plotted on the ordinate. All experiments were performed in triplicate with a β-galactosidase reporter included in the transfection mix for nornalization, with means and one standard deviation indicated by the bars and brackets, respectively. (FIG. 3C) Luciferase reporters containing either four copies of the potential p53 binding sites or mutant versions of these sites were constructed as described in Experimental Procedures. “Min Prom” indicates the minimal promoter present in the vector (pBVLuc). (FIG. 3D) Transfections were performed exactly as in (FIG. 3B) to test the reporters shown in (FIG. 3C).

FIGS. 4A to 4C. JFY1 encodes a mitochondrial protein that interacts with Bcl-2 and Bcl-X_{L}. (FIG. 4A Diagram of expression constructs. For constitutive expression, P_{TK} and P_{CMV} refer to the Herpes Virus thymidine kinase promoter and CMV promoter, respectively. Hyg=hygromycin-B-phosphotransferase gene, conferring resistance to Hygromycin B. For inducible expression, TRE=tetracycline responsive elements, tTA=Tet activator, P_{minCMV}=minimal CMV promoter. This system is activated by removal of Doxycycline (Dox). (FIG. 4B) HA-tagged JFY1 constructs were transfected into 911 cells and visualized by indirect immunofluorescence (green). MitoTracker Red dye was used to visualize mitochondria. JFY1-ΔBH3 encodes a tagged JFY1 protein with a 15 amino acid deletion and is therefore missing the BH3 domain. (FIG. 4C) Different pairs of expression constructs were transfected into 911 cells and total lysates were immunoprecipitated with a rabbit anti-HA antibody, then analyzed by western blotting with the indicated antibodies. The lanes labeled “total lysate” contain ˜25% of the amount of lysate represented in the lanes containing immunoprecipitates.

FIG. 5. JFY1 potently suppresses the growth of human tumor cells. The indicated cell lines were transfected with constructs encoding JFY1, JFY1-ΔBH3, or the empty vector. Cells were harvested 24 hours after transfection and equal cell numbers serially diluted inT25 flasks and grown under selection in hygromycin B for 17 days. Only the highest density flasks are shown. There was no observable difference in colony formation between transfection with JFY1-ΔBH3 and that with the empty vector, while the number of colonies obtained after transfection with the JFY1 expression vector was reduced by more than 1000-fold.

FIGS. 6A to 6E. JFY1 induces rapid apoptosis in DLD1 cells. (FIG. 6A) An expression vector containing separate cassettes for GFP and JFY1 (see FIG. 4A) was used to establish inducible clones of DLD1 cells. Representative results are shown for cells that were maintained in the uninduced state (Off) or after induction by removal of doxycycline from the medium for 12 hours (On). The same fields are shown in the first two columns as viewed under phase contrast (Phase) or fluorescence microscopy (GFP) for the clones that inducibly expresses both GFP and JFY1 (JFY1) or GFP alone (Vector). The third column (DAPI) shows nuclei of the same cell cultures harvested immediately after microscopy and stained with Hoechst 33528. Apoptotic cells stained with this dye have characteristic condensed chromatin and fragmented nuclei. Virtually all JFY1-induced cells were apoptotic by 12 hours. (FIG. 6B) The indicated clones were grown in the presence (Off) or absence (On) of doxycycline for 10 days, then stained with crystal violet. Two different flasks, containing either two million or two thousand cells at the start of the experiment, are shown to illustrate the profound effect of JFY1 induction. (FIG. 6C) DLD1 cells inducibly expressing JFY1 were harvested at the indicated times following doxycycline withdrawal. Whole cell lysates were used in Western blots to assess activation of caspase 9 and cleavage of β-catenin. Cleavage products are indicated by arrows. (FIG. 6D) Identical to FIG. 6C except that the DLD1 cells inducibly expressed p53 instead of JFY1. Note the different time scale. (FIG. 6E) DLD1 cells induced to express either JFY1 or p53 were assayed for apoptosis as indicated by nuclear condensation and fragmentation at the indicated time points. At least 300 cells were counted for each determination, and the experiment was repeated twice with identical results.

### DETAILED DESCRIPTION OF THE INVENTION

It is a discovery of the present inventors that a gene encoding a mitochondrial protein is tightly regulated by p53 and mediates p53-associated apoptosis in CRC cells. In light of the rapid induction of this gene by p53, the gene was named JFY1. The nucleotide sequence of the cDNA is shown in SEQ ID NO: 3 or 4. The encoded amino acid sequence is shown in SEQ ID NO: 1 or 2.

Polynucleotides provided by the present invention include those which are very closely related to SEQ ID NO:3 or 4, including any which encode the same amino acid sequence as shown in SEQ ID NO: 1 or 2. Also included are those which are polymorphic variants of JFY1 as shown, as well as those which are naturally occurring JFY1 mutants and species homologues. Polynucleotide variants typically contain 1, 2, or 3 base pair substitutions, deletions or insertions. Polymorphic protein variants typically contain 1 amino acid substitution, typically a conservative substitution. The percent sequence identity between the sequences of two polynucleotides can be determined using computer programs such as ALIGN which employ the FASTA algorithm, using an affine gap search with a gap open penalty of −12 and a gap extension penalty of −2. According to the present invention, polynucleotides are considered homologues if they achieve at least 90% identity. Preferably they are at least 91%, 93%, 95%, 97%, or even 99% identical. Percent identity between a putative JFY1 polypeptide variant or mutant or homologue can be determined using the Blast2 alignment program. Default settings can be used in comparing the putative sequence to the amino acid sequence of SEQ ID NO: 1 or 2. Preferably they achieve at least 90%, 91%, 93%, 95%, 97%, or even 99%, identity. Polynucleotides preferably comprise at least 730 nucleotides in length of JFY1 coding sequence or at least 1640 nucleotides of total JFY1 transcript or genomic sequence.

Any naturally occurring variants of the JFY1 sequence that may occur in human tissues and which has apoptosis inducing activity are within the scope of this invention. Thus, reference herein to either the nucleotide or amino acid sequence of JFY1 includes reference to naturally occurring variants of these sequences. Nonnaturally occurring variants which differ by as much as four auiino acids and retain biological function are also included here. Preferably the changes are conservative amino acid changes, i.e., changes of similarly charged or uncharged amino acids.

As discussed above, minor amino acid variations from the natural amino acid sequence of JFY1 are contemplated as being encompassed by the term JFY1; in particular, conservative amino acid replacements are contemplated. Conservative replacements are those that take place within a family of amino acids that are related in their side chains. Genetically encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar-glycine, asparagine, glutamnine, cystine, serine, threonine, tyrosine. Phenylalanine, tryptophan, and tyrosine are sometimes classified jointly as aromatic amino acids. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding properties of the resulting molecule, especially if the replacement does not involve an amino acid at a binding site involved in the interaction of JFY1 or its derivatives with a Bcl-2 family member. Whether an amino acid change results in a finctional peptide can readily be determined by assaying the Bcl-2 binding properties of the JFY1 polypeptide derivative. A binding assay is described in detail below. Any members of the family can be used in the assay, although Bcl-2 and Bcl-X_{L} are preferred.

Polynucleotide sequences according to the present invention can be isolated away from other sequences to which they are naturally adjacent in chromosome 19q. Thus they can be isolated away from all or some other human 19q sequences. In a particularly preferred embodiment they are isolated away from all other 19q sequences. The polynucleotides can include a vector for replicating and/or expressing the coding sequence of JFY1. The vector may contain a regulatory sequence which permits control, for example by an inducer or repressor, of expression of JFY1 sequences. Typically the vectors are formed by recombinant in vitro techniques. Vectors can be replicated and maintained in suitable host cells as are known in the art. Pure cultures of the host cells are preferred. Suitable regulatory sequences are known in the art and any such sequence can be used without limitation. The polynucleotide can be joined to another coding sequence, for example, one which encodes an easily assayable epitope or enzyme activity. Such polynucleotides will produce fusion proteins having the properties of both JFY1 and the fusion partner. Fusion proteins can contain all or a part of JFY1 and all or a part of a second protein.

Polynucleotides according to the invention also can be used as primers or probes. Such polynucleotides can be at least 15, 18, 10, or 25 nucleotides in length. They can be double or single stranded. Preferably for use they will be single stranded or denatured. Probes and primers can be labeled using, for example, radiolabels, fluorescent moieties, restriction endonuclease sites, specific hybridization sequences, etc. These can be synthesized according to any technique known in the art for making oligonucleotides. Primer pairs are typically used in tandem and can be packaged together. In one particular embodiment, the primers and/or probes are used to monitor expression of JFY1 as discussed below. Primer pairs of the invention employ at least one primer which is substantially complementary to nucleotides 1-235 of SEQ ID NO:1 or its complement. Substantial complementarity means that the primer will hybridize and initiate template-based extension during amplification.

Polypeptides containing at least 9, 10, 12, 14, 16, or 18 contiguous amino acids of SEQ ID NO: 1 or 2 can be used inter alia to make antibodies. Such polypeptides can be used alone or conjugated or fused to other proteins as immunogens to induce specific binding antibodies to JFY1 in an inoculated animal, such as a mouse, rabbit or goat. Thus polyclonal or monoclonal preparations of JFY1-specific binding antibodies are also provided. Methods for making and screening for such antibodies are well known in the art and can be used by the skilled artisan without recourse to undue experimentation.

Applicants have identified the endogenous control sequences for JFY1 which are found upstream of the coding sequence in the human genome. The control sequences permit binding of p53 which upregulates JFY1 expression. Two such binding sequences were located although one appears to be more active than the other. Either or both of these can be used for coordinately expressing a reporter or other gene sequence with JFY1. The binding sequences can be used with the endogenous coding sequence or with other sequences to exert p53 control. Suitable reporter genes are known in the art, and any can be used including but not limited to Green Fluorescent Protein, β galactosidase, and alkaline phosphatase. The binding sequence can be used singly, or in tandem arrays. Multiple copies increase the level of induction which is achieved. In particular embodiments, a polynucleotide may comprise at least two or at least four copies of the binding sequence. Isolated and purified polynucleotides containing the binding sequences are purified away from other genetic sequences located on chromosome 19q.

Because of JFY1's ability to induce a cell to enter the apoptotic pathway, JFY1 or polynucleotides encoding JFY1 can be used to treat cancers or other diseases characterized by unwanted cellular proliferation. For tumors, the polynucleotide can be administered directly to the tumor or to the body cavity containing the tumor. The polynucleotide can be administered in a virus or in a viral vector. The polynucleotide can be administered in a liposome or other gene delivery particle or formulation. In some situations, the polynucleotide can be delivered by particle bombardment. Those of skill in the art will recognize and be able to match the appropriate delivery method and vehicle for the particular type of tumor or other disease.

Due to the exciting biological activity which JFY1 possesses, it can be used as a basis for drug screening methods. Thus compounds or compositions can be tested by contacting them with a cell which has a mutant p53 and no wild-type p53. JFY1 expression can be monitored, either directly or using a reporter gene under the control of a BS1 (SEQ ID NO:5) and/or BS2 (SEQ ID NO:6 or 27) sequence. A compound or composition which is able to increase JFY1 expression (or surrogate reporter expression) is identified as a candidate for treating cancer or other disease involving cellular proliferation. Monitoring expression can be done by any means known in the art, including measuring a particular protein immunologically or by activity, or by measuring a particular niRNA species. Techniques for measuring expression are well known in the art and any can be used as is convenient. Similar screening techniques can be set up for cell-free systems in which JFY1 expression is monitored, either directly or by surrogate.

Just as p53 can be used diagnostically and prognostically for detection and prediction of cancer disease severity, so can JFY1. Thus a biological sample can be assayed for the amount of an expression product of JFY1. A significantly lower amount in the biological sample than in a control sample identifies a neoplastic sample. Control samples can be obtained from the same individual as the biological sample or it can be obtained from a normal healthy individual. Preferably the control sample will be obtained from the same tissue type as the test sample. If a bona fide tumor sample is tested for expression of JFY1 then a prognosis can be determined. Lower or absent amounts of JFY1 expression products are a negative prognostic indicator, as is lowered expression of p53 in cancer cells.

CRC cell line DLD1 undergoes apoptosis ˜18 hours following expression of exogenous p53 under the control of a doxycycline-regulated promoter. Moreover, these cells are committed to apoptosis after only 9 hours of p53 exposure, as addition of doxycycline after this period does not diminish apoptosis (Yu et al., 1999). These observations, combined with the analysis of numerous p53-regulated genes in this system, led us to propose the following guidelines for candidates that might mediate apoptosis in CRC cells. First, their induction in DLD1 cells should be robust and rapid, with substantial expression by 9 hours. Second, they should be induced by p53 in other CRC lines, not just DLD1 cells. Third, they should be induced not only by high levels of exogenous p53, but also by elevated endogenous p53 following exposure to chemotherapeutic drugs. Fourth, their induction after such exposures should depend on an intact p53 gene. Fifth, the candidate genes should exhibit biochemical and physiologic properties that suggest they can directly stimulate apoptosis through a mitochondrial pathway.

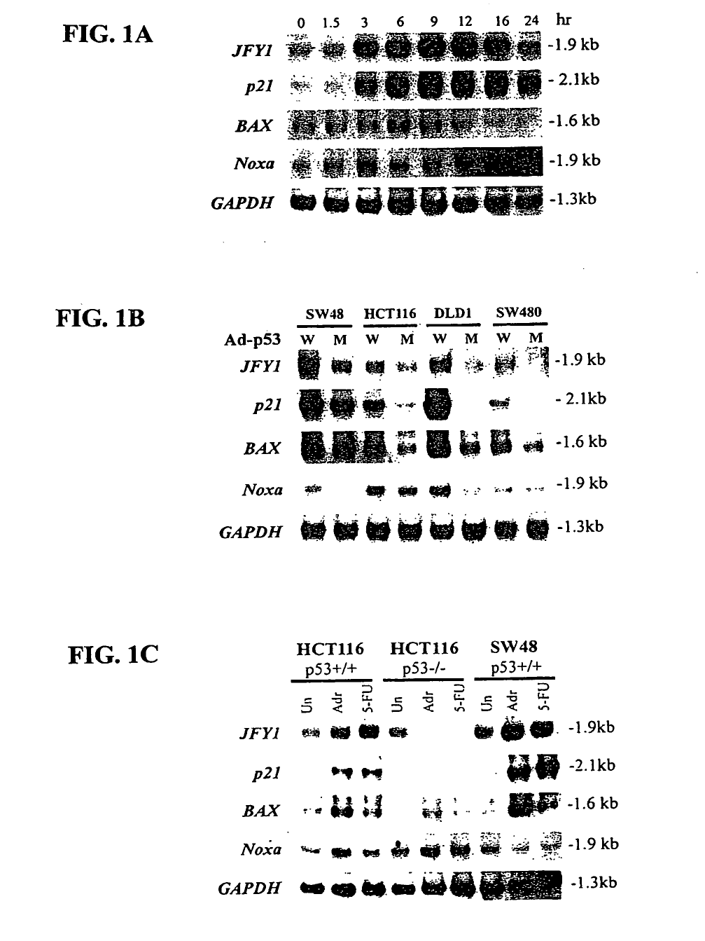

DLD1 cells inducibly expressing p53 were studied using the Serial Analysis of Gene Expression (SAGE) technique (Velculescu et al., 1995; Yu et al., 1999). We identified only one gene, denoted JFY1 which met the criteria described above. The JFY1 gene was discovered through a SAGE tag that matched to ESTs (Expressed Sequence Tags) but to no known genes. The SAGE data indicated that JFY1 was induced over ten-fold in DLD1 cells following p53 expression for 9 hours. Northern blotting showed that JFY1 was induced as soon as 3 hours following doxycycline withdrawal, just as was p21^{CIP1/WAF1} (FIG. 1A). JFY1 expression was maximal by 6 hours, well before the 9-hour “commitment point” for apoptosis determined previously (Yu et al., 1999). In each of four lines tested, there was significant induction of JFY1 after infection with an adenovirus encoding wild type (wt) p53 but none after expression of an analogous adenovirus encoding a mutant R175H p53 (FIG. 1B). Furthermore, JFY1 mRNA expression was found to be induced in HCT116 and SW48 cells following treatment with 5-FU (5-fluorouracil), the mainstay of treatment for CRC, as well as by the DNA-amaging agent adriamycin (FIG. 1C). HCT116 and SW48 cells contain wt p53 genes, and the results in FIG. 1C demonstrate that endogenous levels of p53 were sufficient to induce JFY1. The apoptosis following 5-FU treatment is totally dependent on intact p53 (Bunz et al., 1999). Using HCT116 cells in which the p53 genes had been disrupted by targeted homologous recombination (Bunz et al., 1998), we found that the transcriptional induction of JFY1 by 5-FU was also entirely dependent on p53 (FIG. 1C).

The transcriptional patterns noted above were compared with those of the three other p53-induced genes encoding mitochondrial proteins (BAX, Noxa, and p53AIP1). SAGE revealed only a slight 6r insignificant induction of BAX and Noxa transcripts, as confirmed by Northern blotting (FIG. 1A). p53AIPI transcripts were not detectable by either SAGE or Northern blotting in these experiments, consistent with previous results showing that this gene is activated only at very late times following p53 induction (Oda et al., 2000b). Furthermore, only JFY1 was induced in all four CRC lines tested after infection with adenoviruses, and only JFY1 was significantly induced by 5-FU in both HCT116 and SW48 cells (FIGS. 1B, 1C). In general, the transcriptional patterns of JFY1 closely matched those of p₂₁^{CIP1/WAF1}, while those of the other three genes were considerably different.

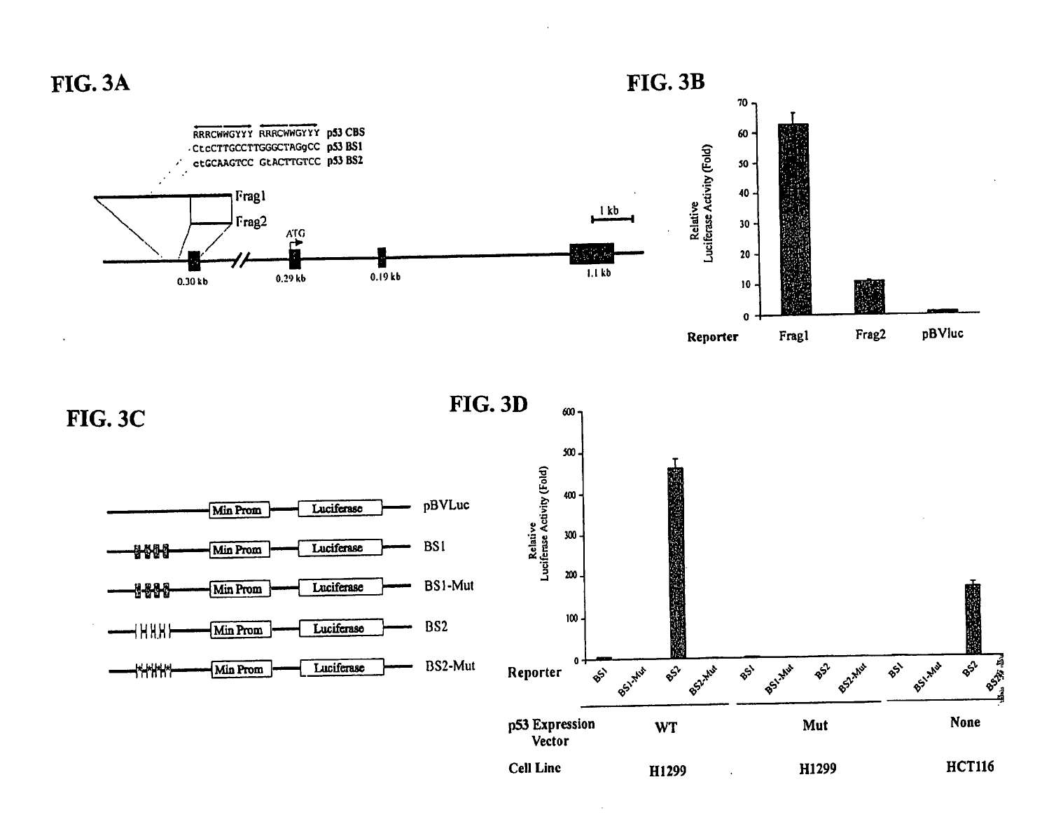

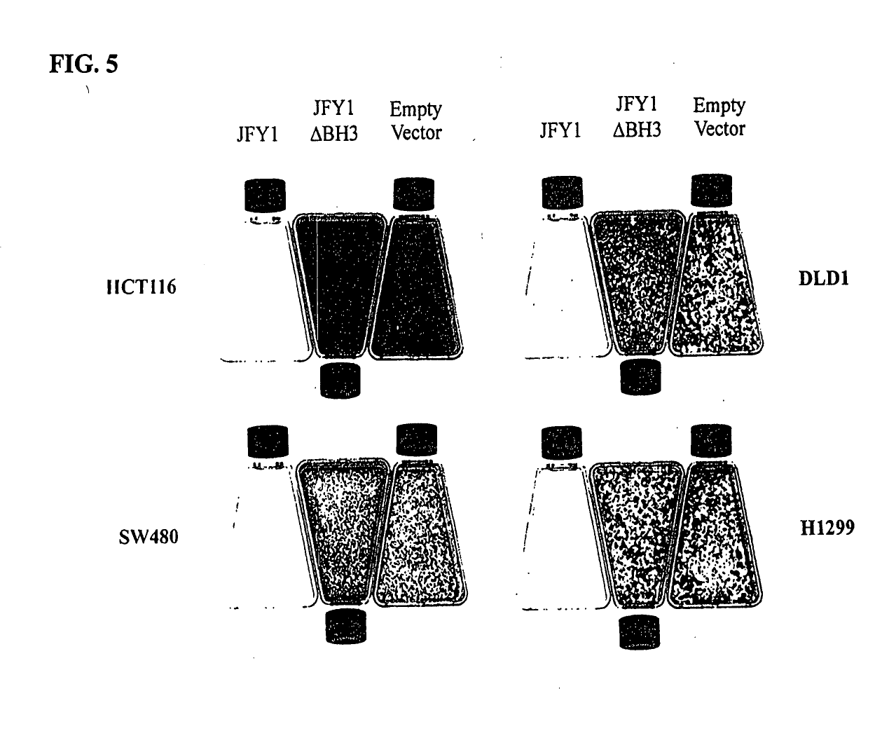

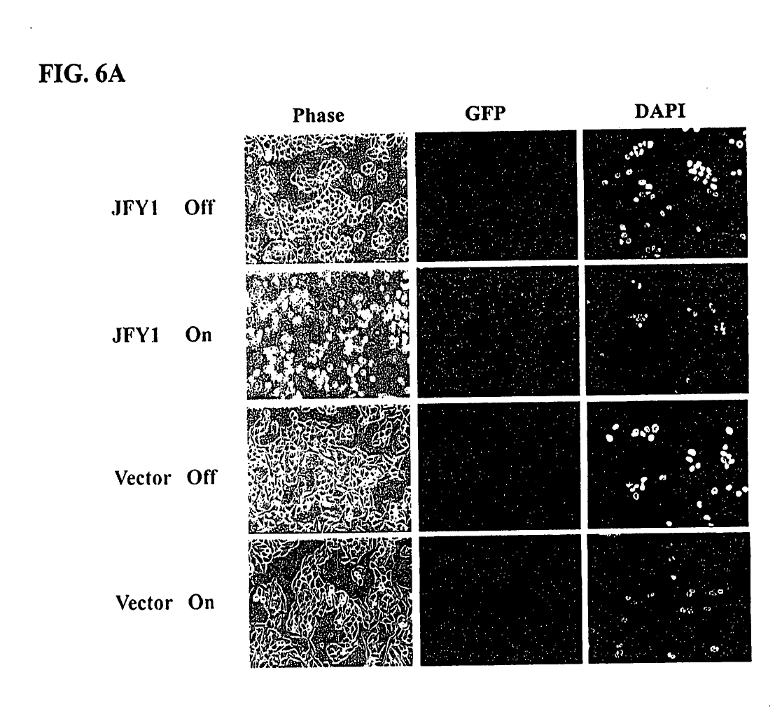

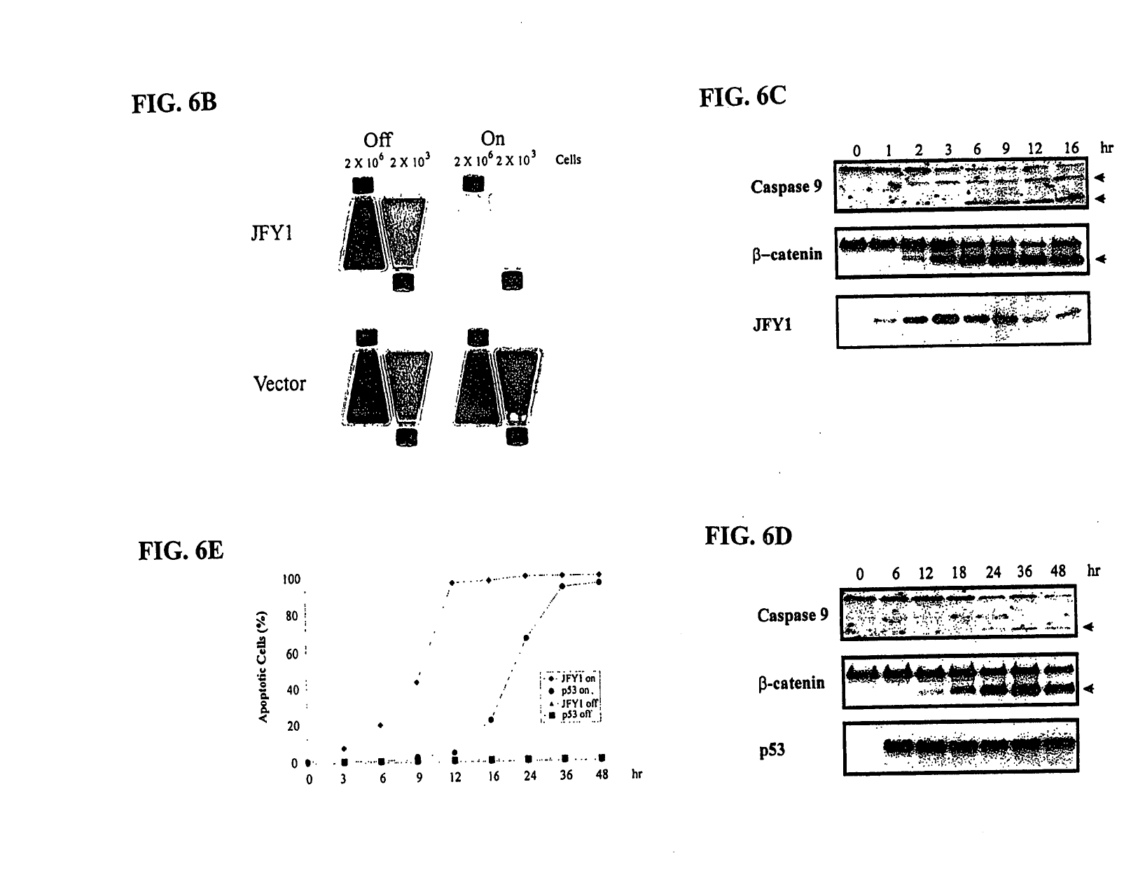

These results suggest that p53-mediated cell death in colorectal cancer cells is in part mediated through the transcriptional activation of the JFY1 gene. The results in FIG. 3 show that this activation is likely the direct result of p53 binding to the BS2 sequences within the JFY1 promoter. The time course of induction of JFY1 (FIG. 1A) and the ability of JFY1 to cause a rapid and profound degree of apoptosis (FIGS. 5, 6) support this model. It is also supported by a large body of literature showing that Bcl-2 family members, particularly those containing only BH3 domains, control apoptotic processes in organisms ranging from C. elegans to humans (Green, 2000; Korsmeyer, 1999; Adams and Cory, 1998; Reed, 1997; Vander Heiden and Thompson, 1999). Finally, it is supported by previous studies showing that p53-mediated apoptosis proceeds through a mitochondrial death pathway (Li et al., 1999; Polyak et al., 1997; Schuler et al., 2000; Soengas et al., 1999).

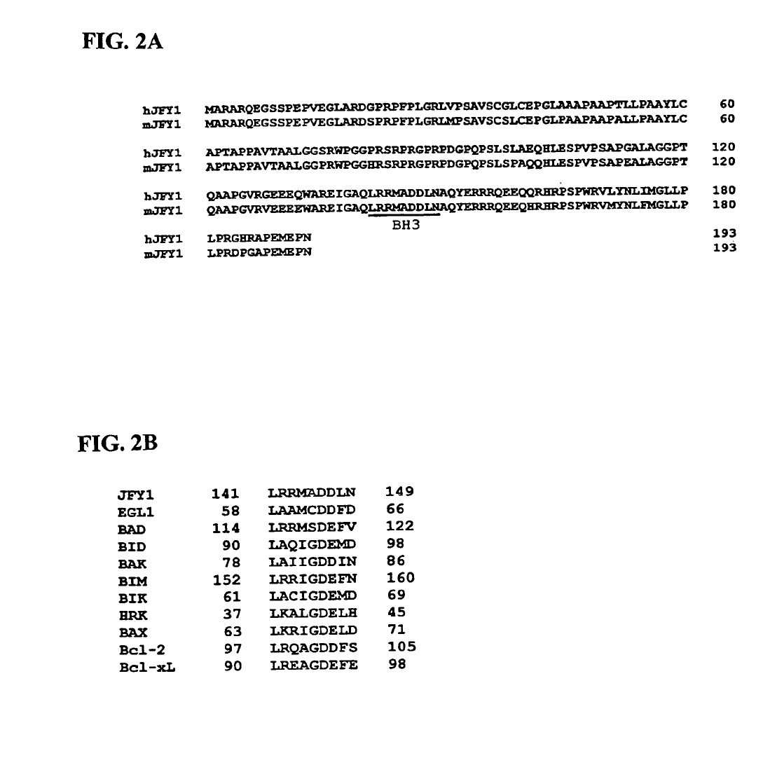

The pore forming abilities of Bcl-2 family members have been documented (Minn et al., 1997; Schendel et al., 1998). JFY1, which is only related to the Bcl-2 family through its BH3 domain, may affect pore formation when complexed with other Bcl-2 family members or with other mitochondrial proteins. Expression of high levels of JFY1 is sufficient for apoptosis, but it is not known whether expression of this gene is necessary for apoptosis. Additionally, JFY1 was expressed, albeit at very low levels, in all normal human tissues analyzed. Targeted deletions of JFY1 in human somatic and mouse ES cells, facilitated by the sequence data provided in FIG. 2, should provide answers to these questions in the future. Finally, the fact that JFY1 expression led to a very rapid and profound apoptosis suggests that it should be considered as a substitute for p53 in cancer gene therapy.

### EXAMPLES

### Example 1

### Characterization of the JFY1 Transcript and Gene

A combination of database searching, re-sequencing of EST clones, RT-PCR analyses, and 5′RACE was used to obtain an apparently full length cDNA for JFY1 (FIG. 2A). These efforts were complicated by an extremely GC rich 5′untranslated region. The final assembled cDNA was 1.9 kb in size, consistent with the size of the major induced transcript observed in Northern blots (FIG. 1A). Comparison of the resultant sequences with that of genomic DNA revealed that the JFY1 transcript was contained within four exons, with the presumptive initiation codon in exon 2 (FIG. 3A). JFY1 was predicted to encode a 193 amino acid protein with no significant homologies to other known proteins except for the BH3 domain discussed below. RT-PCR analysis showed that JFY1 was expressed at low but similar levels in each of eight different human tissues, and radiation hybrid mapping showed that the JFY1 gene is located on chromosome 19q (data not shown).

The mouse homolog of JFY1 was identified through searches of mouse EST and genomic databases. The deduced murine gene contains four exons corresponding to the four coding exons of the human homolog, and the corresponding coding exons were of identical length in the two species. The human and murine genes were 91% and 90% identical at the amino acid and nucleotide levels, respectively (FIG. 2A).

An alternatively spliced form (AS) of JFY1 devoid of exon 2 appeared in some RT-PCR experiments with human RNA templates and likely corresponded to the shorter mRNA species observed in FIG. 1A. Sequencing of PCR products showed that the AS altered the open reading frame so that it no longer contained a BH3 domain, and we therefore did not evaluate this form further.

We searched for consensus p53-binding sites upstream of the JFY1 gene and identified two such sites, BS1 and BS2, lying 230 and 144 bp upstream of the transcription start site, respectively (FIG. 3A). To determine whether this region of the JFY1 gene could mediate p53-responsiveness, we cloned a 493 bp fragment whose 5′ end was 427 bp upstream of the putative transcription start site, and placed it in front of a luciferase reporter containing a minimal promoter. Inclusion of this region conferred a 60-fold activation when transfected into H1299 cells together with a p53 expression vector (FIG. 3B). Deletion of the 5′ terminal 300 bp from this construct (a region which contained BS1 and BS2), led to loss of most of the p53 responsiveness (FIG. 3B).

To determine which of the two binding sites was primarily responsible for the p53 responsiveness, we tested constructs containing four copies of either binding site, in wt or mutant form, inserted upstream of a luciferase reporter and minimal promoter (FIG. 3C). In the mutant forms, two residues predicted to be critical for p53 binding were substituted with non-cognate nucleotides. These experiments revealed that BS2 was likely to be the major p53 responsive element, as it was activated over 400-fold by exogenous p53 in H1299 cells, while BS1 was activated only 7-fold (FIG. 3D). Co-transfection of the BS2 reporter with a mutant p53 R175H expression vector did not result in reporter activation (FIG. 3D). Additionally, mutation of the BS2 sequence completely abrogated wt p53 responsiveness (FIG. 3D). Finally, we transfected the BS2 reporter into HCT116 cells, which contain endogenous wt p53, in the absence of an exogenous p53 expression vector. Transfection of the BS2 reporter, but not the BS1 or mutant BS2 reporters, resulted in high levels of luciferase activity in these experiments, suggesting that endogenous levels of p53 are sufficient for direct JFY1 activation (FIG. 3D). BS2 was also conserved in the murine JFY1 gene.

### Example 2

### JFY1 Encodes a BH3 Domain-Containing Mitochondrial Protein that Interacts with BcL-2 and Bcl-X_{L}

Two observations led us to test the hypothesis that JFY1 encoded a mitochondrial protein. First, the JFY1 protein was predicted to contain a BH3 domain (FIG. 2B). BH3 domains are one of the four Bcl-2 homology domains present in Bcl-2 family of proteins (Chittenden et al., 1995). Several of the pro-apoptotic members of this family contain the BH3 domain but not the BH1, 2, or 4 domains and reside at least partially in mitochondria (reviewed in (Korsmeyer, 1999; Reed, 1997)). The BH3 domains are essential for their pro-apoptotic activities and for their ability to heterodimerize with other Bcl-2 family members (Wang et al., 1998; Wang et al., 1996; Zha et al., 1997). Second, a GenBank entry (Accession U82987) corresponding to a partial JFY1 cDNA sequence carried the intriguing annotation of “Human Bcl-2 binding component 3”. The basis for this annotation was not specified and the amino acid sequence included with this entry was out of frame with respect to the major protein we predicted to be encoded by the JFY1 gene.

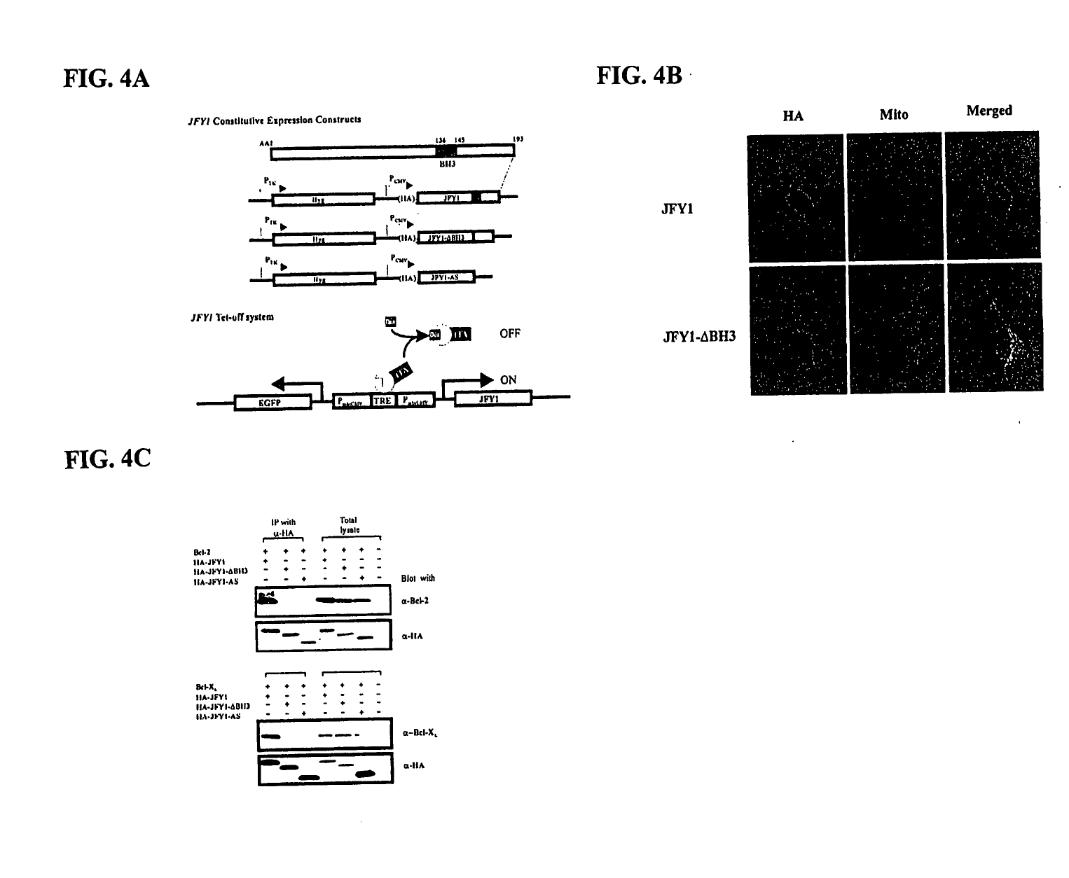

To determine the subcellular localization of human JFY1, we constructed an expression vector encoding the full length JFY1 protein with an amino-terminal hemaglutanin (HA) tag (FIG. 4A). This vector was expressed in 911 cells, which have a flat morphology that facilitates subcellular localization studies. Indirect immunofluorescence with an anti-HA antibody showed punctate perinuclear staining in all transfected cells (FIG. 4B). Comparison of this localization with that of a dye that labeled mitochondrial membranes (MitoTracker Red) indicated complete colocalization (FIG. 4A). Interestingly, the BH3 domain was not required for this localization, as the protein generated from another JFY1 expression vector, JFY1-ΔBH3, (identical except for the deletion of the BH3 domain), was also found exclusively in mitochondria (FIG. 4B). This lack of dependence on BH3 for mitochondrial localization is consistent with data on other BH3-containing proteins, though it distinguished JFY1 from Noxa, in which the BH3 domain was required (Oda et al., 2000a).

We next tested whether JFY1 interacted with Bcl-2. Using the JFY1 expression vector described above, we expressed JFY1 together with Bcl-2 in 911 cells. Inmunoprecipitation experiments showed that a major fraction of Bcl-2 (˜50%) was bound to JFY1 under these conditions (FIG. 4C). The BH3 domain of JFY1 was essential for this interaction, as deletion of the BH3 domain completely abrogated the binding (FIG. 4C). A similar vector encoding the alternatively spliced (AS) form of JFY1 provided an additional control in this experiment (FIG. 4C).

Previous experiments have shown that Bcl-2 is not expressed in many CRCs, while Bcl-X_{L} is ubiquitously expressed (Zhang et al., 2000). To determine whether JFY1 also binds to Bcl-X_{L}, 911 cells were co-transfected with JFY1 plus Bcl-X_{L} expression vectors and analogous immunoprecipitation experiments performed. As shown in FIG. 4C, Bcl-X_{L} bound to intact JFY1 and the BH3 domain of JFY1 was essential for this binding.

### Example 3

### JFY1 Expression Results in Complete and Rapid Cell Death

To determine the effect of JFY1 expression on cell growth, we constructed an expression vector containing JFY1 plus a Hygromycin B resistance gene (FIG. 4A) and transfected it into four different cancer cell lines. Following selection, there was a drastic reduction in colony formation following transfection with the JFY1 expression vector compared to the empty vector or to an analogous vector encoding JFY1 without its BH3 domain (FIG. 5). This colony suppression was observed regardless of the p53 genotype of the cells (wt in HCT116 cells, mutant in SW480 and DLD1, null in H1299). Enumeration showed that JFY1 expression reduced colony formation by over 1000-fold.

For comparison, we analyzed the time course of caspase activation and apoptosis following p53 expression in DLD1 cells. Though expression of p53 and JFY1 were induced immediately upon doxycycline withdrawal (FIGS. 6C, 6D and data not shown), it took several hours longer for caspase 9 activation and β-catenin degradation to appear following p53 expression (note the different time scales in FIGS. 6C and 6D). Moreover, morphological signs of apoptosis, such as condensed chromatin and fragmented nuclei, appeared ˜9 hours later in cells expressing p53 compared to cells expressing JFY1 (FIG. 6E).

### Example 4

### Experimental Procedures

### Cell Culture

The human colorectal cancer cell lines DLD-1, HCT116, SW48, SW480 and the human lung cancer cell line H1299 were obtained from ATCC. HCT 116 cells with a targeted deletion of p53 has been previously described (Bunz et al., 1998). All lines were maintained in McCoy's 5A media (Life Technologies) supplemented with 10% fetal bovine serum (HyClone), 100 units/ml of penicillin and 100 ug/ml of streptomycin at 37° C. The retinal epithelial cell line 911 was kindly provided by A. J. Van der Eb of the University of Leiden and maintained as described (Fallaux et al., 1996). Chemotherapeutic agents were used at concentrations of 0.2 ug/ml (adriamycin) and 50 ug/ml (5-FU) and cells were treated for 24 hours. Transfections were performed with Fugene™ 6 (Boehringer Mannheim) according to the manufacturer's instructions.

### Constructs

JFY1 expression plasmids: The HA-tagged, full length JFY1 expression vector pHAHA-JFY1 was constructed by cloning RT-PCR products into the pCEP4 vector (Invitrogen). Variants of this vector containing JFY1 with the BH3 domain deleted, or the alternatively spliced form of JFY1, were constructed similarly. Sequences for the primers and details of vector construction are available from authors upon request. In all cases, inserts of multiple individual clones were completely sequenced and the ones that were free of mutation were subsequently used for experiments. The Bcl-2 expression vector was described previously (Pietenpol et al., 1994) and the V5-tagged Bcl-X_{L} expression vector was purchased from Invitrogen.

### Reporter Constructs and Reporter Assay

Promoter-containing fragments were amplified from human genomic DNA of HCT116 cells and cloned into the pBVLuc luciferase reporter vector containing a minimal promoter (He et al., 1998). To test presumptive p53-binding sites, the following oligo pairs containing two copies of wildtype or mutant binding sites were used: 5′-CTAGGCTCCTTGCCTTGGGCTAGGCCACACTCTCCTTGCCTTGGGCTAGGCC-3′ (SEQ ID NO: 18) and 5′-CTAGGGCCTAGCCCAAGGCAAGGAGA GTGTGGCCTAGCCCAAGGCAAGGAGC-3′ (SEQ ID NO: 19) for BS1, 5′-CTAGGCTCATTACCTTGGGTTAAGCCACACTCTCATTACCTTGGGTTAAGC C-3′ (SEQ ID NO: 20) and 5′-CTAGGGCTTAACCCAAGGTAATGAG AGTGTGGCTTAACCCAAGGTAATGAGC-3′ (SEQ ID NO: 21) for BS1mut, 5′-CTAGGCTGTAAGTTCCTGAATTATCCACACTCTGCAAGTTCCTGAATTGTCC-3′ (SEQ ID NO: 22) and 5′-CTAGGGACAAGTCAGGACTTGCAGA GTGTGGACAAGTCAGGACTTGCAGC-3′ (SEQ ID NO: 23) for BS2, 5′-CTAGGCTGTAATTCCTGAATTATCCACACTCTGTAATTCCTGAATTATCC-3′ (SEQ ID NO: 24) and 5′-CTAGGGATAATTCAGGAATTACAGA GTGTGGATAATTCAGGAATTACAGC-3′ (SEQ ID NO: 25) for BS2mut. The annealed oligonucleotide pairs were concatamerized and cloned into the Nhe I site of pBVLuc. Transfections of 911 cells were performed in 12-well plates using 0.2 ug luciferase reporter plasmid, 0.2 ug pCMVβ and 0.8 ug pCEP4 encoding either wt p53 or mutant p53R175H. The β-galactosidase reporter pCMVβ Promega) was included to control for transfection efficiency. Luciferase and β-galactosidase activities were assessed 24-48 hours following transfection with reagents from Promega and ICN Pharmaceuticals, respectively. All reporter experiments were performed in triplicate and repeated on at least three independent occasions. Transfections with HCT116 cells were performed similarly except that 0.4 ug luciferase reporter and 0.4 ug β-galactosidase reporter were used for each well, without p53 expression vectors.

### Inducible Cell Lines

The method for generating inducible cell lines in DLD1 cells has been previously described (Yu et al., 1999). In brief, the HA-tagged full length JFY1 cDNA was cloned into pBi-MCS-GFP to create pBi-JFY1-GFP. Linearized pBi-JFY1-GFP and pTK-hyg (Clontech) were co-transfected into DLD1-TET cells at a molar ratio of 5 to 1. DLD1-TET cells are DLD1 derivatives containing a constitutively expressed tet activator (Gossen and Bujard, 1992; Yu et al., 1999). Single colonies were obtained by limiting dilution in the presence of 400 ug/ml G418, 250 ug/ml Hygromycin B (Calbiochem), and 20 ng/ml doxycycline for 3-4 weeks. Clones that had low background GFP fluorescence and homogeneous GFP induction were selected and analyzed for the expression of JFY1 by western blot analysis.

### Immunoprecipitation and Western Analysis

Immunoprecipitation was performed essentially as described (Chan et al., 1999) with the following modifications. 911 cells were seeded in T75 flasks 18 hours prior to transfection with 5 ug of each of two expression constructs (10 ug total) and harvested 20 hours after transfection. The cell suspension was sonicated for 15 seconds in a total volume of 1 ml and incubated with 30 ul protein A:protein G beads (4:1, Boehringer Mannheim) for one hour at 4° C. The supernatants collected after centrifugation (“total lysates”) were subsequently used for immunoprecipitation with rabbit antibody against HA (sc-805, Santa Cruz). Western blotting of total lysates and immunoprecipitates were performed as previously described (Chan et al., 1999). Other antibodies used in these experiments included a mouse monoclonal antibody against hemagglutinin (12CA, Boehringer Mannheim), a rabbit antibody against caspase-9 (sc-7890 Santa Cruz), a mouse monoclonal antibody against Bcl-2 (OP60, Oncogene Sciences), a mouse monoclonal antibody against V5, (R960-25, Invitrogen), a mouse monoclonal antibody against β-catenin (C19220, Transduction labs), and a mouse monoclonal antibody against p53 (DO1, gift of D. Lane).

### Immunofluorescence and Confocal Microscopy

911 cells were seeded on glass chamber slides (Nalge Nunc, Lab-Tek 177372) and transfected with JFY1 expression constructs. Twenty hours later, MitoTracker Red (0.5 uM, Molecular Probes) was added to the medium and the cells were incubated at 37° C. for an additional 20 minutes. Cells were fixed with 4% paraformaldehyde in PBS, perneablized with cold acetone and blocked with 100% goat serum for 1 hour at room temperature. After three washes in PBST (PBS with 0.05% Tween-20), slides were incubated with anti-HA antibody (12CA, Boehringer Mannheim) diluted 1:200 with 50% goat serum in PBST at 4° C. overnight. After four washes in PBST for 5 min each, slides were incubated with Alexa⁴⁸⁸conjugated anti-mouse antibody (A-11001, Molecular Probes) diluted 1:250 in PBST for 30 minutes at room temperature. After four additional washes in PBST, slides were mounted and analyzed by confocal microscopy.

### Cell Growth and Apopt Sis Assays

Approximately 1×10⁶ cells were plated in each T25 flask 18 to 24 hours prior to transfection. Twenty four hours following transfection with constitutive JFY1 expression constructs, cells were harvested by trypsinization and serial dilutions were plated in T25 flasks under hygromycin selection (0.1 mg/ml for HCT116, 0.25 mg/ml for DLD1 and 0.4 mg/ml for SW480 and H1299). Attached cells were stained with crystal violet 14 to 17 days later. For DLD1 lines containing inducible JFY1 constructs, cells were grown in doxycycline and serially diluted in T25 flasks. Twenty-four hours after seeding, the medium was replaced with fresh growth media with or without doxycycline and cells were allowed to grow for 10 days, and then stained with crystal violet. To determine the fraction of apoptotic cells, all cells (attached and floating) were collected and stained with Hoechst 33258 as described (Waldman et al., 1996). Cells with characteristic condensed chromatin and fragmented nuclei were scored as apoptotic.

### Northern Blot Analysis

Total RNA was prepared using RNAgents (Promega) and 10 ug of total RNA was separated by electrophoresis in 1.5% formaldehyde agarose gels. Probes for Northern blotting were generated by PCR using cellular cDNA or ESTs as template and labeled by random priming (Feinberg and Vogelstein, 1984). The sequences of the primers used to prepare all probes are available from authors upon request. Northern blot analysis was performed and hybridized in QuickHyb (Stratagene) as described (Zhang et al., 1997).

### REFERENCES

Adams, J. M., and Cory, S. (1998). The Bcl-2 protein family: arbiters of cell survival, Science 281, 1322-1326.

Baker, S. J., Markowitz, S., Fearon, E. R., Willson, J. K., and Vogelstein, B. (1990). Suppression of human colorectal carcinoma cell growth by wild-type p53, Science 249, 912-915.

Brodsky, M. H., Nordstrom, W., Tsang, G., Kwan, E., Rubin, G. M., and Abrams, J. M. (2000). Drosophila p53 binds a damage response element at the reaper locus, Cell 101, 103-13.

Bunz, F., Dutriaux, A., Lengauer, C., Waldman, T., Zhou, S., Brown, J. P., Sedivy, J. M., Kinzier, K. W., and Vogelstein, B. (1998). Requirement for p53 and p21 to sustain G2 arrest after DNA damage, Science 282, 1497-501.

Bunz, F., Hwang, P. M., Torrance, C., Waldman, T., Zhang, Y., Dillehay, L., Williams, J., Lengauer, C., Kinzler, K. W., and Vogelstein, B. (1999). Disruption of p53 in human cancer cells alters the responses to therapeutic agents, J Clin Invest 104, 263-9.

Chan, T. A., Hermeking, H., Lengauer, C., Kinzier, K. W., and Vogelstein, B. (1999). 14-3-3Sigma is required to prevent mitotic catastrophe after DNA damage, Nature 401,616-20.

Chao, C., Saito, S., Kang, J., Anderson, C. W., Appella, E., and Xu, Y. (2000). p53 transcriptional activity is essential for p53-dependent apoptosis following DNA damage, Embo J 19,4967-4975.

Chittenden, T., Flemington, C., Houghton, A. B., Ebb, R. G., Gallo, G. J., Elangovan, B., Chinnadurai, G., and Lutz, R. J. (1995). A conserved domain in Bak, distinct from BH1 and BH2, mediates cell death and protein binding functions, Embo J 14, 5589-96.

El-Deiry, W. S. (1998). Regulation of p53 downstream genes, Semin Cancer Biol 8, 345-57.

El-Deiry, W. S., Kern, S. E., Pietenpol, J. A., Kinzler, K. W., and Vogelstein, B. (1992). Definition of a consensus binding site for p53, Nat Genet 1, 45-9.

El-Deiry, W. S., Tokino, T., Velculescu, V. E., Levy, D. B., Parsons, R., Trent, J. M., Lin, D., Mercer, W. E., Kinzler, K. W., and Vogelstein, B. (1993). WAF1, a potential mediator of p53 tumor suppression, Cell 75, 817-25.

Fallaux, F. J., Kranenburg, O., Cramer, S. J., Houweling, A., Van Ormondt, H., Hoeben, R. C., and Van Der Eb, A. J. (1996). Characterization of 911: a new helper cell line for the titration and propagation of early region 1-deleted adenoviral vectors, Hum Gene Ther 7, 215-22.

Feinberg, A. P., and Vogelstein, B. (1984). “A technique for radiolabeling DNA restriction endonuclease fragments to high specific activity”. Addendum, Anal Biochem 137, 266-7.

Gossen, M., and Bujard, H. (1992). Tight control of gene expression in mammalian cells by tetracycline-responsive promoters, Proc Natl Acad Sci USA 89, 5547-51.

Gottlieb, T. M., and Oren, M. (1998). p53 and apoptosis, Semin Cancer Biol 8, 359-68.

Green, D. R. (2000). Apoptotic pathways: paper wraps stone blunts scissors, Cell 102, 1-4.

Harper, J. W., Adami, G. R., Wei, N., Keyomarsi, K., and Elledge, S. J. (1993). The p21 Cdk-interacting protein Cip1 is a potent inhibitor of G1 cyclin-dependent kinases, Cell 75, 805-16.

He, T. C., Sparks, A. B., Rago, C., Hermeking, H., Zawel, L., da Costa, L. T., Morin, P. J., Vogelstein, B., and Kinzler, K. W. (1998). Identification of c-MYC as a target of the APC pathway, Science 281, 1509-12.

Hermeking, H., Lengauer, C., Polyak, K., He, T.-C., Zhang, L., Thiagalingam, S., Kinzler, K. W., and Vogelstein, B. (1997). 14-3-3s is a p53-regulated inhibitor of G2/M progression, Molecular Cell 1, 3-11.

Hollstein, M., Hergenhahn, M., Yang, Q., Bartsch, H., Wang, Z. Q., and Hainaut, P. (1999). New approaches to understanding p53 gene tumor mutation spectra, Mutat Res 431, 199-209.

Hussain, S. P., and Harris, C. C. (1999). p53 mutation spectrum and load: the generation of hypotheses linking the exposure of endogenous or exogenous carcinogens to human cancer, Mutat Res 428, 23-32.

Jin, S., Martinek, S., Joo, W. S., Wortman, J. R., Mirkovic, N., Sali, A., Yandell, M. D., Pavletich, N. P., Young, M. W., and Levine, A. J. (2000). Identification and characterization of a p53 homologue in Drosophila melanogaster, Proc Natl Acad Sci USA 97, 7301-6.

Korsmeyer, S. J. (1999). BCL-2 gene family and the regulation of programmed cell death, Cancer Res 59, 1693s-1700s.

Lane, D. P. (1999). Exploiting the p53 pathway for cancer diagnosis and therapy, Br J Cancer 80 Suppl 1, 1-5.

Levine, A. J. (1997). p53, the cellular gatekeeper for growth and division, Cell 88, 323-31.

Li, P. F., Dietz, R., and von Harsdorf, R. (1999). p53 regulates mitochondrial membrane potential through reactive oxygen species and induces cytochrome cindependent apoptosis blocked by Bcl-2, Embo J 18, 6027-36.

Minn, A. J., Velez, P., Schendel, S. L., Liang, H., Muchmore, S. W., Fesik, S. W., Fill, M., and Thompson, C. B. (1997). Bcl-x(L) forms an ion channel in synthetic lipid membranes, Nature 385, 353-7.

Oda, E., Ohki, R., Murasawa, H., Nemoto, J., Shibue, T., Yamashita, T., Tokino, T., Taniguchi, T., and Tanaka, N. (2000a). Noxa, a BH3-only member of the Bcl-2 family and candidate mediator of p53-induced apoptosis, Science 288, 1053-8.

Oda, K., Arakawa, H., Tanaka, T., Matsuda, K., Tanikawa, C., Mori, T., Nishimori, H., Tamai, K., Tokino, T., Nakamura, Y., and Taya, Y. (2000b). p53AIP1, a potential mediator of p53-dependent apoptosis, and its regulation by Ser-46-phosphorylated p53, Cell 102, 849-62.

Ollmann, M., Young, L. M., Di Como, C. J., Karim, F., Belvin, M., Robertson, S., Whittaker, K., Demsky, M., Fisher, W. W., Buchman, A., et al. (2000). Drosophila p53 is a structural and functional homolog of the tumor suppressor p53, Cell 101, 91-101.

Oren, M. (1999). Regulation of the p53 tumor suppressor protein, J Biol Chem 274, 36031-4.

Pietenpol, J. A., Papadopoulos, N., Markowitz, S., Willson, J. K., Kinzler, K. W., and Vogelstein, B. (1994). Paradoxical inhibition of solid tumor cell growth by bcl2, Cancer Res 54, 3714-7.

Polyak, K., Xia, Y., Zweier, J. L., Kinzler, K. W., and Vogelstein, B. (1997). A model for p53 induced apoptosis, Nature 389, 300-304.

Prives, C., and Hall, P. A. (1999). The p53 pathway, J Pathol 187, 112-26.

Reed, J. C. (1997). Double identity for proteins of the Bcl-2 family, Nature 387, 773-6.

Reed, J. C. (1999). Dysregulation of apoptosis in cancer, J Clin Oncol 17, 2941-53.

Schendel, S. L., Montal, M., and Reed, J. C. (1998). Bcl-2 family proteins as ion-channels, Cell Death Differ 5, 372-80.

Schuler, M., Bossy-Wetzel, E., Goldstein, J. C., Fitzgerald, P., and Green, D. R. (2000). p53 induces apoptosis by caspase activation through mitochondrial cytochrome c release, J Biol Chem 275, 7337-42.

Soengas, M. S., Alarcon, R. M., Yoshida, H., Giaccia, A. J., Hakem, R., Mak, T. W., and Lowe, S. W. (1999). Apaf-1 and caspase-9 in p53-dependent apoptosis and tumor inhibition, Science 284, 156-9.

Symonds, H., Krall, L., Remington, L., Saenz-Robles, M., Lowe, S., Jacks, T., and Van Dyke, T. (1994). p53-dependent apoptosis suppresses tumor growth and progression in vivo, Cell 78, 703-11.

Vander Heiden, M. G., and Thompson, C. B. (1999). Bcl-2 proteins: regulators of apoptosis or of mitochondrial homeostasis?, Nat Cell Biol 1, E209-E216.

Velculescu, V. E., Zhang, L., Vogelstein, B., and Kinzler, K. W. (1995). Serial Analysis Of Gene Expression, Science 270, 484-487.

Waldman, T., Lengauer, C., Kinzler, K. W., and Vogelstein, B. (1996). Uncoupling of S phase and mitosis induced by anticancer agents in cells lacking p21, Nature 381, 713-16.

Wang, K., Gross, A., Waksman, G., and Korsmeyer, S. J. (1998). Mutagenesis of the BH3 domain of BAX identifies residues critical for dimerization and killing, Mol Cell Biol 18, 6083-9.

Wang, K., Yin, X. M., Chao, D. T., Milliman, C. L., and Korsmeyer, S. J. (1996). BID: a novel BH3 domain-only death agonist, Genes Dev 10, 2859-69.

Xiong, Y., Hannon, G. J., Zhang, H., Casso, D., Kobayashi, R., and Beach, D. (1993). p21 is a universal inhibitor of cyclin kinases, Nature 366, 701-704.

Yu, J., Zhang, L., Hwang, P. M., Rago, C., Kinzler, K. W., and Vogelstein, B. (1999). Identification and classification of p53-regulated genes, Proc Natl Acad Sci USA 96, 14517-22.

Zha, J., Harada, H., Osipov, K., Jockel, J., Waksman, G., and Korsmeyer, S. J. (1997). BH3 domain of BAD is required for heterodimerization with BCL-XL and pro-apoptotic activity, J Biol Chem 272, 24101-4.

Zhang, L., Yu, J., Park, B.-H., Kinzier, K. W., and Vogelstein, B. (2000). Role of BAX in the Apoptotic Response to Anti-cancer Agents, Science 290, In press.

Zhang, L., Zhou, W., Velculescu, V. E., Kern, S. E., Hruban, R. H., Hamilton, S. R., Vogelstein, B., and Kinzler, K. W. (1997). Gene Expression Profiles in Normal and Cancer Cells, Science 276, 1268-1272.

We claim:

1. An isolated and purified JFY1 protein having the sequence shown in SEQ ID NO: 1 or 2.

2. An isolated and purified JFY1 coding sequence having the sequence shown in SEQ NO: 3 or 4.

3. A vector comprising the coding sequence of claim 2.

4. The vector of claim 3 in which the JFY1 coding sequence is transcriptionally regulated by an exogenous inducer or repressor.

5. An isolated and purified JFY1 BS1 or BS2 nucleic acid having the sequence shown in SEQ ID NO: 5, 6, or 27.

6. The isolated and purified nucleic acid of claim 5 which is operably linked to a reporter gene such that p53 regulates transcription of the reporter gene.

7. A method of inducing apoptosis in cancer cells, comprising:
supplying a nucleic acid comprising a JFY1 coding sequence to cancer cells, whereby JFY1 is expressed and induces apoptosis in said cancer cells.

8. A method of screening drugs for those which can induce apoptosis, comprising:
contacting a test compound with a cell comprising a mutant p53 and no wildtype p53;
detecting JFY1 expression, wherein a test compound which increases JFY1 expression is a candidate drug for treating cancer.

9. A method of screening drugs for those which can induce apoptosis, comprising:
contacting a test compound with a cell comprising a mutant p53 and a JFY1-BS2-reporter gene construct, said cell comprising no wild-type p53;
detecting reporter gene expression, wherein a test compound which increases reporter gene expression is a candidate drug for treating cancer.

10. The method of claim 7 wherein the step of supplying is intratumoral.

11. The method of claim 7 wherein the JFY1 coding sequence is in a viral vector.

12. The method of claim 7 wherein the JFY1 coding sequence is supplied in a liposome.

13. The isolated and purified JFY1 BS2 nucleic acid of claim 5 which has at least two copies of BS2.

14. The isolated and purified JFY1 BS2 nucleic acid of claim 5 which has at least four copies of BS2.

15. An isolated and purified JFY1 protein which is at least 90% identical to the sequence of SEQ ID NO: 1 or 2.

16. An isolated and purified JFY1 coding sequence which is at least 90% identical to the sequence of SEQ ID NO: 3 or 4.

17. A method for diagnosing cancer cells, comprising the step of:
assaying an expression product of JFY1 in a biological sample suspected of being neoplastic;
comparing amount of the expression product in the biological sample to amount of the expression product in a control sample which is not neoplastic;
identifying the biological sample as neoplastic if the amount of the expression product in the biological sample is significantly less than the amount in the control sample.

18. The method of claim 17 wherein the control sample and the biological sample are obtained from a single individual.

19. The method of claim 18 wherein the control sample and biological sample are obtained from the same tissue type.

20. A method to aid in determining prognosis of a cancer patient, comprising the step of:
assaying an expression product of JFY1 in a tumor sample;
comparing amount of the expression product in the tumor sample to amount of the expression product in a control sample which is not neoplastic;
identifying the biological sample as having a negative prognostic indicator if the amount of the expression product in the tumor sample is significantly less than the amount in the control sample.

21. The method of claim 20 wherein the control sample and the tumor sample are obtained from a single individual.

22. The method of claim 21 wherein the control sample and tumor sample are obtained from the same tissue type.

23. The method of claim 20 wherein the control sample and biological sample are obtained from the same tissue type.

24. An isolated and purified polypeptide comprising at least 9 contiguous amino acids of a JFY1 protein as shown in SEQ ID NO: 1 or 2.

25. The polypeptide of claim 24 which comprises at least 15 of said contiguous amino acids.

26. A fusion protein which comprises at least 9 contiguous amino acids of a JFY1 protein as shown in SEQ ID NO: 1 or 2 covalently bonded to at least an epitope of a non-JFY1 protein.

27. The fusion protein of claim 26 which comprises a complete non-JFY1 protein.

28. The fusion protein of claim 26 which comprises a complete JFY1 protein.

29. A host cell comprising a vector according to claim 3.

30. The host cell of claim 29 which is in a pure culture.

31. An isolated and purified polynucleotide which comprises at least 1640 contiguous nucleotides of SEQ ID NO:3 or 4 or the complement thereof.

32. The polynucleotide of claim 31 which is labeled with a detectable moiety.

33. An isolated and purified polynucleotide which comprises at least 18 contiguous nucleotides selected from nucleotides 1-235 of SEQ ID NO:1.

34. The polynucleotide of claim 33 which comprises nucleotides 1-235 of SEQ ID NO:1.

35. A pair of two oligonucleotides which can be used as primers for amplifying a JFY1 coding sequence, wherein each of said two oligonucleotides hybridizes to a distinct strand of JFY1 and wherein at least one of said pair of oligonucleotides hybridizes to nucleotides 1-235 of SEQ ID NO:1 or its complement.


SEQUENCE LISTING (30 sequences)

SEQ ID NO: 1 - protein, 193 aa, Homo sapiens
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 2 - protein, 193 aa, Mus musculus
MARARQEGSSPEPVEGLARDSPRPFPLGRLMPSAVSCSLCEPGLPAAPAAPALLPAAYLCAPTAPPAVTAALGGPRWPGGHRSRPRGPRPDGPQPSLSPAQQHLESPVPSAPEALAGGPTQAAPGVRVEEEEWAREIGAQLRRMADDLNAQYERRRQEEQHRHRPSPWRVMYNLFMGLLPLPRDPGAPEMEPN

SEQ ID NO: 3 - DNA, 1912 bp, Homo sapiens
gcggcgcgagccacatgcgagcgggcgcctggcggcggcggcggcggcaccagcgatcccagcagcggccacgacgcggacgcgcctgcggcccggggagcagcagcagccacagccacagcagccgccactgcagttagagcggcagcagcagcgacagccacagcagcagccgccgcggagagcggcgctcggcgggcgcgccctcctgaaggaagccgcccgccccccaccgccgccccctccggcgtgttcatgcccccggggccccagggagcgccatggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattaggtgcctgcacccgcccggtggacgtcagggactcggggggcaggcccctcccacctcctgacaccctggccagcgcgggggactttctctgcaccatgtagcatactggactcccagccctgcctgtcccgggggcgggccggggcagccactccagccccagcccagcctggggtgcactgacggagatgcggactcctgggtccctggccaagaagccaggagagggacggctgatggactcagcatcggaaggtggcggtgaccgagggggtggggactgagccgcccgcctctgccgcccaccaccatctcaggaaaggctgttgtgctggtgcccgttccagctgcaggggtgacactgggggggggggggctctcctctcggtgctccttcactctgggcctggcctcaggcccctggtgcttccccccctcctcctgggagggggcccgtgaagagcaaatgagccaaacgtgaccactagcctcctggagccagagagtggggctcgtttgccggttgctccagcccggcgcccagccatcttccctgagccagccggcgggtggtgggcatgcctgcctcaccttcatcagggggtggccaggaggggcccagactgtgaatcctgtgctctgcccgtgaccgccccccgccccatcaatcccattgcataggtttagagagagcgacgtgtgaccactggcattcatttggggggtgggagattttggctgaagccgccccagccttagtccccagggccaagcgctggggggaagacggggagtcagggagggggggaaatctcggaagagggaggagtctgggagtggggagggatggcccagcctgtaagatactgtatatgcgctgctgtagataccggaatgaattttctgtacatgtttggttaattttttttgtacatgatttttgtatgtttccttttcaataaaatcagattggaacagtgaaaaaaaaaaaaaaagggcggccgctcagagtatccctcgaggggcccaacgttacgcgtacccagctt

SEQ ID NO: 4 - DNA, 2091 bp, Mus musculus
atgcgagcggggagcccaggaggcggcggcgacaccagcaagcaagcagcagcagcggtgatccggacacgaagactccagaagcagcagcagtcactgcagttagagcagcaggagcagcagcaaggtgcctcaatagcaacccactcggcgggcgagccctccagaaggcaaccgcccgccaccccatcgcctcctttctccggagtgttcatgcccccggggctccagggagcgccatggcccgcgcacgccaggagggcagctctccggagcccgtagagggtctagcccgcgacagtccgcgccccttcccgctcggccgcctgatgccctccgctgtatcctgcagcctttgcgagcccggcctgcccgccgcccctgctgcccctgccttgctgccggccgcctacctctgcgcccccaccgctccacctgccgtcaccgccgccctggggggcccccgctggcctgggggtcaccgcagccggcccagaggcccgcgcccggacggtcctcagccctccctgtcaccagcccagcagcacttagagtcgcccgtgcccagcgccccggaggccctggcaggaggccccacccaagctgccccgggagtgcgtgtggaggaggaggagtgggcccgggagatcggggcccagctgcggcggatggcggacgacctcaacgcgcagtacgagcggcggagacaagaagagcagcatcgacaccgaccctcaccctggagggtcatgtacaatctcttcatgggactcctccccttacccagggatcctggagccccagaaatggagcccaactaggtgcctacacccgcccgggggacgtcggagacttggggggcaggaccccctccgccttctgacaccctggccagcgcgggggactttttctgcaccatgtagcatactggactgccagccttgcccgtcccaggggcaggcaagggatgccactcgagcccgggcagcctgggtgcactgatggagatacggacttggggggaccctggcctcccgaaagccagggaagggagggctgaaggactcatggtgactgagggggtggggaccgagccgcccgcctctgccgcccaccaccatctcaggaaaggctgctggtgctggctgcccgttccagctgcaggggggacgctgggggtgtccccagtgcgccttcactttgggcctggcctcaggcccctggtgcttccccccctcctcctgaggagggggtctgtgaagagcatatgagccaaacctgaccactagcctcctggagccagagaatggggggcgtgtgaaggccttcttaacccagtgcccagccatcttccctgagccgccggcgggcggtgaacgatgcctgcctcaccttcatctgggggtgtccaggaggggtccagactgtgaatcctgtgctctgcccgggaccaccccccccccccaatccccatccatctcattgcataggtttagagagagcacgtgtgaccactggcattcatttggggggtgggagatattggcggaagccaccccagccttagtccccagggcaaagcgctggggaggaagatggggagtcagggaggggggaagtctcagaagagggaggagtctgggagcggggagggacggcccagcctgtaagatactgtacatgcactgctgtagatatactggaatgaattttctgtacatgtttggttaattttttttgtacatgatttttgtatgtttccttttcaataaaatcagattgaacagtgaacactctttttgttagctttaccagtgacagagcatctggcactacctgtaaggacatgaaagaaacggtgtgtgtgtgtatgtgtgtgtgtgtgtgtgtgtgtgtgtgtgagaaatggctcagtggttaagagcactgactgctcttccagaggtcctgagttcaaatcccagcaaccacatggtggctcacaaccatcataatgagatcagacaccctcttctggagtgtctgaaggcagctacagtgtacttacatataacaataaataaatgtaaaaaagagaaagaaagaaagaaa

SEQ ID NO: 5 - DNA, 21 bp, Homo sapiens
ctccttgccttgggctaggcc

SEQ ID NO: 6 - DNA, 20 bp, Homo sapiens
ctgcaagtcctgacttgtcc

SEQ ID NO: 7 - protein, 9 aa, Homo sapiens
LRRMADDLN

SEQ ID NO: 8 - protein, 9 aa, Homo sapiens
LAAMCDDFD

SEQ ID NO: 9 - protein, 9 aa, Homo sapiens
LRRMSDEFV

SEQ ID NO: 10 - protein, 9 aa, Homo sapiens
LAQIGDEMD

SEQ ID NO: 11 - protein, 9 aa, Homo sapiens
LAIIGDDIN

SEQ ID NO: 12 - protein, 9 aa, Homo sapiens
LRRIGDEFN

SEQ ID NO: 13 - protein, 9 aa, Homo sapiens
LACIGDEMD

SEQ ID NO: 14 - protein, 9 aa, Homo sapiens
LKALGDELH

SEQ ID NO: 15 - protein, 9 aa, Homo sapiens
LKRIGDELD

SEQ ID NO: 16 - protein, 9 aa, Homo sapiens
LRQADDDFS

SEQ ID NO: 17 - protein, 9 aa, Homo sapiens
LREAGDEFE

SEQ ID NO: 18 - DNA, 52 bp, Homo sapiens
ctaggctccttgccttgggctaggccacactctccttgccttgggctaggcc

SEQ ID NO: 19 - DNA, 52 bp, Homo sapiens
ctagggcctagcccaaggcaaggagagtgtggcctagcccaaggcaaggagc

SEQ ID NO: 20 - DNA, 52 bp, Homo sapiens
ctaggctcattaccttgggttaagccacactctcattaccttgggttaagcc

SEQ ID NO: 21 - DNA, 52 bp, Homo sapiens
ctagggcttaacccaaggtaatgagagtgtggcttaacccaaggtaatgagc

SEQ ID NO: 22 - DNA, 50 bp, Homo sapiens
ctaggctgcaagtcctgacttgtccacactctgcaagtcctgacttgtcc

SEQ ID NO: 23 - DNA, 50 bp, Homo sapiens
ctagggacaagtcaggacttgcagagtgtggacaagtcaggacttgcagc

SEQ ID NO: 24 - DNA, 50 bp, Homo sapiens
ctaggctgtaattcctgaattatccacactctgtaattcctgaattatcc

SEQ ID NO: 25 - DNA, 50 bp, Homo sapiens
ctagggataattcaggaattacagagtgtggataattcaggaattacagc

SEQ ID NO: 26 - DNA, 19 bp, Homo sapiens
rrrcwwgyyrrrcwwgyyy

SEQ ID NO: 27 - DNA, 20 bp, Homo sapiens
ctgcaagccccgacttgtcc

SEQ ID NO: 28 - protein, 242 aa, Homo sapiens
PPPPACSCPRGPRERHGPRTPGGQLPGARRGPGPRRPAPLPARPPGALGSVLRPLRARPGCRPRRPHPAARCLPLRPHRPTRRHRRPGGFPLAWGSPQPAPRPAPGRSSALALAGGAAPGVARAQRPGGSGGRSHPGGPGSPRGGGTVGPGDRGPAAADGGRPQRTVRAAETRGAAAAPPLTLEGPVQSHHGTPALTQGPQSPRDGAQLGACTRPVDVRDSGGRPLPPPDTLASAGDFLCTM

SEQ ID NO: 29 - DNA, 495 bp, Homo sapiens
gcgagactgtggccttgtgtctgtgagtacatcctctgggctctgcctgcacgtgactttgtggaccctggaacgcccgtcggtcggtctgtgtacgcatcgctgggggtgtggatctgtgggtcccagtcagtgtgtgtgtccgactgtcccggtgtctgggcgatctccccacaccccgccgcacagcgcctgggtcctccttgccttgggctaggccctgccccgtcccccgctgcagggaaacccccggcgcggaggtaggggggggcgcggcgcgcgcctgcaagtcctgacttgtccgcggcgggcgggcggggccgtagcgtcacgcgggggcggggcgtgggacccgccgggcgggggcggggcggggcggggcggggcggctttggagcgggcccgggatccgccgggcggcctgagacgcggcgcgagccacatgcgagcgggcgcctggcggcggcggcggcggcaccagcgatcccagcagcg

SEQ ID NO: 30 - DNA, 581 bp, Mus musculus
gcccttgtcctgatgtgtatctgtgcctctggtctgactttgtgtccctgtggctcagtcatcactgactcagtgcaccctggcgtgccagtccgttagtctgagcgtactcctcaggtgtgggtgtgggtcccagtcagtgtgtcagtgtgtcaagcgtgtgtccggacaccctaggtctgggctgtccccacgctgctcctcctgcctggaccaggcctcgccccgcccctctggctgccgggaaaccccccgcgcccgaggtagggggcgcggcgcccgactgcaagccccgacttgtccccagccgcgggcggggccctggcgtcacgcgggggcggggcgtgggagccagcgagaggcggggcggggcggccgccgagcgagcggggcccggggatctgccgggaggcctgagacgcggcatagagccacatgcgagcggggagcccaggaggcggcggcgacaccagcaagcaagcagcagcagcggtgatccggacacgaagactccagaagcagcagcagtcactgcagttagagcagcaggagcagcagcaaggtgcctcaatagcaaccca